(19) United States
(12) Patent Application Publication
(10) Pub. No.: US 2013/0251658 A1
(43) Pub. Date: Sep. 26, 2013

(54) HYDROPHOBIZED PROTEIN HYDROLYSATE

(71) Applicants: Martin Schilling, Bonn (DE); Oliver Thum, Ratingen (DE)

(72) Inventors: Martin Schilling, Bonn (DE); Oliver Thum, Ratingen (DE)

(73) Assignee: EVONIK GOLDSCHMIDT GMBH, Essen (DE)

(21) Appl. No.: 13/992,097

(22) PCT Filed: Nov. 10, 2011
(86) PCT No.: PCT/EP2011/069811

§ 371 Date: Jun. 6, 2013

(30) Foreign Application Priority Data
Dec. 8, 2010 (DE) 10 2010 062 600.7

(51) Int. Cl. C12P 21/06

(52) U.S. Cl. CPC C12P 21/06
USPC 424/70.14; 514/1.1; 435/68.1; 514/18.8; 516/16; 516/9; 510/501

(57) ABSTRACT

The invention relates to enzymatically hydrophobicised protein hydrolysates, their production and use, and to cosmetic preparations comprising these.

### FIELD OF THE INVENTION

The invention relates to enzymatically hydrophobicised protein hydrolysates, their production and use, and also cosmetic preparations comprising these.

### PRIOR ART

Proteins and protein hydrolysates are an interesting class of raw material because they are readily available, of natural origin and biodegradable. However, due to the lack of lipophilic groups, the interface-active properties of the proteins are only weakly marked. It is therefore necessary to introduce additional lipophilic groups in order to obtain efficient, protein-based, interface-active compounds. According to the prior art, the desired modified proteins have been produced for a relatively long time by condensation of protein hydrolysates and acid chlorides (hereinbelow PHFSK) and, on account of their surface-active properties, are used in various applications (e.g. for producing emulsions, foams, conditioning of skin and hair, dispersing of pigments in a variety of solvents etc.).

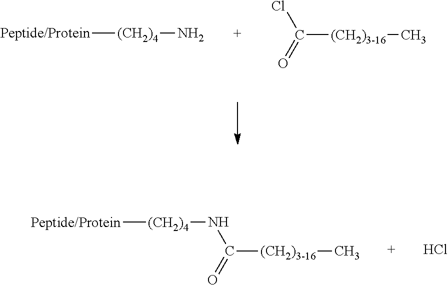

However, the isolation of the acid chlorides required for the synthesis (e.g. with thionyl chloride from fatty acid mixtures) is unacceptable from an ecological point of view, and moreover a not inconsiderable amount of salt is produced during the condensation reaction; this can considerably alter the product properties and therefore has to be separated off, possibly involving high expenditure. Furthermore, the degrees of modification obtained are very low, or can be achieved only with large excesses of acid (Roussel-Philippe et al., European Journal of Lipid Science and Technology 102[2], 97-101. 2000). Finally, the derivatisation alters the repertoire of available basic groups since the primary amino groups of the lysine radicals are preferentially amidated. This is disadvantageous since the presence of cationic groups is desired in certain applications, such as e.g. hair conditioning, but the lysine radicals are no longer available after amidation.

The use of transglutaminases (TG) for the crosslinking of proteins and protein hydrolysates has been known for a relatively long time. The reaction is a transamidation reaction between lysine and glutamine radicals of proteins during which ammonia is released and an isopeptide bond is formed.

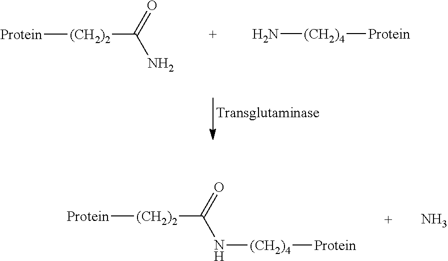

Transglutaminase catalysed crosslinking reaction of proteins

It is likewise known that primary alkylamines can also serve as nucleophile instead of the lysine in the transglutaminase catalysed transamidation reaction. This has been shown in the literature for a synthetic dipeptide as model substrate (Ohtsuka et al., (2000) J. Agric. Food Chem 48:6230-6233) and for intact proteins (Nieuwenhuizen et al., (2004) Biotechnol Bioeng 85:248-258).

Taking only the structures of the starting materials and products into consideration, then it is formally an alkylation of the peptide. Consequently, in connection with the present invention, the discussion is of “alkylated protein hydrolysates” or “alkylation”.

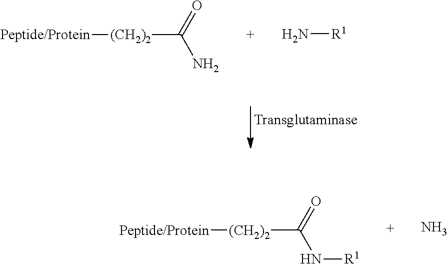

### Transglutaminase Catalysed Alkylation of Proteins

In the case of the intact proteins, however, compared to the PHFSKs, only a low degree of modification (g of alkyl radicals per g of protein) could be achieved (6-8% for PHFSKs, 0.2-0.4% for TG catalysed alkylation, cf. Ohtsuka et al., (2000) J. Agric. Food Chem 48:6230-6233, Nieuwenhuizen et al., (2004) Biotechnol Bioeng 85:248-258 and Roussel-Philippe et al., European Journal of Lipid Science and Technology 102[2], 97-101. 2000).

Consequently, the amphiphilic character of these reaction products is only weakly marked and these proteins hydrophobicised by means of transglutaminase catalysis cannot serve as a replacement for the classic PHFSKs.

It was an object of the invention to provide hydrophobicised peptides which overcome at least one disadvantage of the prior art.

### DESCRIPTION OF THE INVENTION

Surprisingly, it has been found that the protein hydrolysates alkylated with the help of transglutaminases and described below are able to achieve the object of the invention.

The present invention therefore provides enzymatically hydrophobicised protein hydrolysates, their production and use.

The invention further provides formulations, in particular cosmetic preparations, comprising hydrophobicised protein hydrolysates according to the invention.

One advantage of the present invention is that no acid chlorides are needed during the synthesis.

It is a further advantage that the products can have, on account of the sometimes very high glutamine content of the protein hydrolysates, a high degree of derivatisation and, at the same time, smaller concentrations of alkylamines have to be used since, in contrast to the acid chlorides, secondary reactions (hydrolysis) do not occur.

As a result, an exceptional foaming behaviour can advantageously be observed. It is also an advantage that, as by-product, instead of sodium chloride (in the case of the acid chlorides), ammonia is produced, which can be driven off easily. Moreover, the reaction proceeds in the neutral pH range, meaning that salt is also not introduced on account of pH adjustments. Finally, it is an important advantage that the amino groups of the protein hydrolysate remain largely intact since in the presence of the alkylamines primarily the glutamine radicals are converted by means of transglutaminase catalysis. The primary amino groups of the protein hydrolysates (terminal and the ε-amino group of the lysine) make a considerable contribution to the physicochemical properties of the products (e.g. to the interaction/binding with/to negatively charged surfaces of skin and hair=substantivity). During the derivatisation with acid chlorides, these nucleophilic amino groups are preferably converted to amides and can thus no longer contribute to the substantivity.

The present invention provides an alkylated peptide mixture obtainable by a process involving the process steps
A) hydrolysis of at least one protein comprising at least one glutamine radical to give a protein hydrolysate and optionally purification of the protein hydrolysate,
B) bringing the protein hydrolysate into contact with a transglutaminase and at least one primary or secondary amine of the general formula (I), in particular a primary amine,

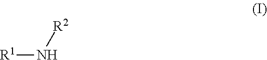

where R¹ and R², independently of one another, is identical or different and is selected from an optionally unsaturated, optionally substituted, optionally branched organic radical having 5 to 40, preferably 5 to 22, in particular 6 to 18, carbon atoms,
C) purification of the alkylated peptide mixture.

In connection with the present invention, the term “alkylated peptide mixture” is to be understood as meaning a mixture comprising at least two peptides alkylated on in each case at least one glutamine. Hence, it is evident that for the case where, in process step A), only the protein of one type is hydrolysed, this protein must have at least two glutamine radicals.

Unless stated otherwise, all stated percentages (%) are percentages by mass.

In practice, it is advantageous to use readily available protein sources in process step A); these are accessible in particular as mixtures of proteins. Consequently, the at least one protein is preferably selected from the list comprising, preferably consisting of:

isolated plant storage proteins such as e.g. wheat protein (gluten), soya protein, pea protein, rice protein, maize protein, lupin protein,
animal proteins such as e.g. collagen, keratin, casein, whey proteins (lactoglobulins), silk protein (fibroin) and
microbial proteins such as e.g. yeast protein extracts, algae proteins or bacterial biomass (SCP=single cell protein).

It is advantageous according to the invention to use in process step A) a mixture of proteins which has a high fraction of glutamine radicals, hence the protein is preferably selected from the list comprising, preferably consisting of, wheat gluten, isolated storage proteins of legumes, in particular soya, pea, lupin and milk proteins, in particular caseins and lactoglobulins, with wheat gluten being very particularly preferred.

The hydrolysis in process step A) is preferably catalysed by adding acid, particularly preferably through the use of enzymes. Suitable processes are known to the person skilled in the art; likewise, it requires no effort to adjust the respective process parameters in such a way that protein hydrolysates with desired average molecular weights are formed in process step A). Instructions of this kind for enzymatically catalysed hydrolysis processes can be found by a person skilled in the art in Aaslyng et al., (1988) J. Agric. Food Chem 46:481-489 and Adler-Nissen J (1976) J. Agric. Food Chem 24:1090-1093, for processes catalysed by acid or alkali in Aaslyng et al., (1998) J Agric. Food Chem 46:481-489.

According to the invention, the protein hydrolysate from process step A) preferably has an average molecular weight of from 203 g/mol to 100 000 g/mol, preferably from 500 g/mol to 20 000 g/mol, in particular from 1000 g/mol to 15 000 g/mol.

Protein hydrolysates which can be produced according to process step A) and used directly in process step B) are also commercially available; these are for example: Meripro 810 and Meripro 711 (wheat protein hydrolysates, enzymatically and chemically, Syral), Naturalys® W (wheat protein hydrolysate, Roquette), Cropeptide W, Hydrotriticum 2000, Tritisol, Tritisol XM (wheat protein hydrolysates with different molecular weight distributions, Croda), Hydrosoy 2000 (soya protein hydrolysate, Croda), Gluadin® W20 and Gluadin® WLM (wheat protein hydrolysates with different molecular weight distributions, Cognis), AMCO HCA411 and HLA-198 (casein or whey protein hydrolysate, American Casein Company).

In process step B), in principle all transglutaminases belonging to EC class 2.3.2.13 known to the person skilled in the art can be used, as can a fragment of these trans-glutaminases which has corresponding enzyme activity. Such enzymes can be isolated, for example, from Streptoverticillium, Bacillus, various Actinomycetes and Myxomycetes, but also from plants, fish and mammal sources, such as, for example, pig liver. EP 2 123 756 and WO2009101762 describe transglutaminases stabilised compared to their wild type, the use of which is preferred in process step B) according to the invention.

It is preferred that the transglutaminases used in process step B) can be isolated selected from the list consisting of Bacillus subtilis, Streptomyces mombaraensis (formerly Streptoverticillium mobaraense). In this connection, a particularly preferred transglutaminase is selected from the transglutaminases from Ajinomoto available under the trade name “Activa” (transglutaminases from Streptomyces mombaraensis), e.g. Activa®WM, Activa®EB, Activa®PB, Activa®WS, Activa®YG.

In process step B), the protein hydrolysate is preferably used in a concentration of between 5% by weight and 40% by weight, preferably between 15% by weight and 25% by weight, based on the total reaction mixture, where the solvent and/or dispersant is preferably water. In some circumstances, it may be advantageous to heat the reaction mixture to above 50° C., preferably to above 70° C., in particular to above 80° C., before adding the transglutaminase in process step B) in order to increase the rate of the hydration process, during which thorough mixing, for example by stirring, of the reaction mixture preferably takes place.

The radicals R¹ and R² of the primary or secondary amine of the general formula (I) in process step B) are preferably selected from the group consisting of alkyl and alkenyl radical, preferably linear, unsubstituted alkyl and alkenyl radical, in particular an octyl, decyl, dodecyl, tetradecyl, hexadecyl, octadecyl, octadecenyl, octadecadienyl, eicosyl, docosyl radical.

The radicals R¹ and R² can also be mixtures of alkyl radicals, in particular technical-grade mixtures of these alkyl radicals. The alkyl radicals of such technical mixtures are preferably derived from fatty acid mixtures which can be obtained by various processes from vegetable fatty acid mixtures and can be fractionated by means of various processes. The fatty acid composition of such vegetable fatty acid mixtures varies depending on the oil seed used for the isolation and is known to the person skilled in the art for example in the form of optionally fractionated rapeseed oil, soya oil, sunflower oil, tallow oil, coconut oil fatty acids.

Consequently, the alkylamines preferably used in process step B) are selected from the group consisting of: rapeseed fatty amine, soya fatty amine, sunflower fatty amine, tallow fatty amine, palm fatty amine, palm kernel fatty amine and coconut fatty amine. The amine of the general formula (I) is used in process step B) preferably in a concentration between 0.25% by weight and 10% by weight, in particular between 0.5% by weight and 5% by weight, based on the total reaction mixture. The enzyme activity in the reaction mixture in process step B) is preferably 10-25 000 U/I, preferably 200-1000 U/I, particularly preferably 300-600 U/I, where the unit U can be determined in accordance with the hydroxamate assay described in Folk and Cole (1966), Biochim. Biophys. Acta 122:244-264.

In process step B), the pH is preferably between 5 and 10, preferably between 6 and 8, particularly preferably between 6.5 and 7.5.

Preferably, a thorough mixing, for example by stirring, of the reaction mixture takes place during the transglutaminase reaction in process step B).

The temperature of the reaction mixture during the transglutaminase reaction in process step B) is preferably 20° C. to 50° C., preferably 30° C. to 45° C. and particularly preferably 35° C. to 40° C.

The reaction time of the transglutaminase reaction in process step B) is up to several hours depending on the temperature used.

If, in process step A), the hydrolysis is catalysed by the use of at least one enzyme, it may be advantageous, for time-saving reasons, if process step A) and process step B) are carried out simultaneously.

The present invention further provides the process described above with which peptide mixtures according to the invention can be produced. Processes preferred according to the invention are those which lead to the aforementioned preferred peptide mixtures according to the invention.

The alkylated peptide mixtures according to the invention can be used advantageously in cleaning compositions, in cosmetic or pharmaceutical formulations, and also in crop protection formulations.

Consequently, the present invention further provides cosmetic, dermatological or pharmaceutical formulations, crop protection formulations, and also care and cleaning compositions and surfactant concentrates comprising alkylated peptide mixtures according to the invention.

The term “care composition” is understood here as meaning a formulation which satisfies the purpose of retaining an object in its original form, of reducing or avoiding the effects of external influences (e.g. time, light, temperature, pressure, soiling, chemical reaction with other reactive compounds that come into contact with the object) such as, for example, ageing, soiling, material fatigue, bleaching, or even of improving desired positive properties of the object. For the last point, mention may be made for example of improved hair shine or greater elasticity of the object under consideration.

“Crop protection formulations” are to be understood as meaning those formulations which are obviously used for crop protection depending on the nature of their preparation; this is the case especially if at least one compound from the classes of herbicides, fungicides, insecticides, acaricides, nematicides, protectants against birds, plant nutrients and soil structure improvers is present in the formulation. Cosmetic compositions preferred according to the invention are selected from the group consisting of: creams, lotions, rinses and shampoos.

Peptide mixtures according to the invention have advantageous emulsifying and foam-stabilising properties. A further subject matter of the present invention is therefore a use of the peptide mixtures according to the invention as emulsifier, such as, for example, O/W or W/O emulsifier, as conditioner for skin and hair, as dispersion auxiliary, in particular for cosmetic pigments, as foam former or foam stabiliser.

The present invention is described by way of example in the examples listed below, without any intention of restricting the invention, the scope of application of which arises from the overall description and the claims, to the embodiments specified in the examples.

The following figures form part of the examples:

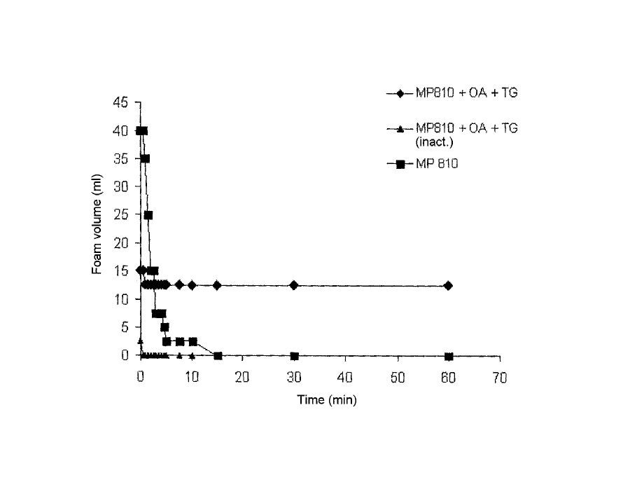

FIG. 1: Improvement in the foam stability through transglutaminase catalysed hydrophobicisation of modified wheat protein hydrolysate. (“MP810” (squares)=Meripro 810, Syral; “MP810+OA+TG (inact.)” (triangles)=Meripro 810+octylamine (OA)+inactivated transglutaminase (TG)=negative control; “MP810+OA+TG” (diamonds)=Meripro 810+octylamine (OA)+transglutaminase (TG)

### EXAMPLES

### Example 1

### Transglutaminase Catalysed Hydrophobicisation of a Wheat Protein Hydrolysate with Octylamine and Laurylamine and Comparison with the Product of Non-Hydrolysed Wheat Protein and Comparison with Non-Hydrophobicised Wheat Protein Hydrolysate

A commercial where protein (Amygluten 110, Syral, molecular weight >200 kD) and a hydrolysate produced from this (Meripro 810, molecular weight ˜10 kD) were in each case dispersed in water at pH=7.5 and a concentration of 10% by weight together with octylamine or laurylamine (2.5% by weight). 1% by weight of a commercial transglutaminase preparation (Activa WM, Ajinomoto) was added and the mixture was stirred at 45° C. over a period of 24 h. The enzyme was then deactivated at a temperature of 80° C. As controls, in each case mixtures with deactivated enzyme and mixtures without alkylamine were carried out. The conversion of the alkylamines was determined by means of a photometric assay following derivatisation with 1-chloro-2,4-dinitrobenzene (CDNB) compared to the control reactions (Ekladius and King, (1957) Biochem J 65:128-131).

Whereas in the case of the reaction with non-hydrolysed wheat protein (Amygluten 110, Syral), no conversion of the alkylamine could be detected, in the case of the hydrolysate, an alkylamine conversion of more than 50% was measured.

Approximately 10% of the amino acid radicals and ˜30% of the available glutamine radicals were modified.

### Example 2

### Surface Activity

The surface tension towards air and/or the interfacial tension towards paraffin and/or diethylhexyl carbonate (DEC) was determined by means of the pendant drop method. Measurements were carried out on 1% strength solutions of the wheat protein hydrolysate modified with octylamine and the corresponding control reactions. As a result of the modification, the interfacial activity could be considerably improved (reduction of interfacial tension and surface tension, Table 1).

TABLE 1
Influence of the transglutaminase catalysed hydrophobicisation
of a wheat protein hydrolysate (Meripro 810, Syral) on the
interfacial activity.

| | Surface | Interfacial tension | Interfacial tension |
|---|---|---|---|
| | tension | towards paraffin | towards |
| | (mN/m) | (mN/m) | DEC (mN/m) |
| Wheat protein | 41 | 4 | 9 |
| hydrolysate + | | | |
| octylamine + inactive | | | |
| transglutaminase | | | |
| Wheat protein | 31 | 2 | 7 |
| hydrolysate + | | | |
| octylamine + active | | | |
| transglutaminase | | | |

### Example 3

### Foam Formation and Foam Stability

The effect of the hydrophobicisation of the wheat protein hydrolysate with octylamine on the foam formation and foam stability was compared in shaking experiments of 1% solutions with the corresponding controls. For this, 10 ml of the corresponding samples were poured into a 50 ml polypropylene centrifuge tube with volume scale and shaken for one minute under identical conditions. The foam volume above the liquid was read off in the course of time in order to assess starting foam volume and foam stability. A considerable foam-stabilising effect of the hydrophobicised wheat protein hydrolysate was found here (FIG. 1).

### Example 4

### Emulsion Performance

The emulsifying properties of the wheat protein hydrolysate modified with octylamine and of the wheat protein hydrolysate modified with laurylamine were investigated on a 1 ml scale in shaking experiments. For a triglyceride/water/emulsifier ratio of 20/79/1, the various samples and controls were investigated. The emulsion was prepared by intensive shaking and the kinetics of the phase separation were monitored. It was found here that the phase separation in the case of the hydrophobicised protein hydrolysates was considerably slower than in the case of the corresponding controls and was incomplete.

### Example 5

### Comparison with an Acid Chloride-Modified Wheat Protein Hydrolysate

A laurylamine-modified wheat protein hydrolysate was synthesised as described in Example 1; the concentration of the laurylamine used was 0.625% (w/w). With the help of acid chloride, a wheat protein hydrolysate modified with lauryl chloride (a PHFSK) was produced, using the same material concentrations and the same wheat protein hydrolysate as during the preparation catalysed by transglutaminase. For the Schotten-Baumann condensation reaction, the procedure was in accordance with optimum reaction conditions described in the literature (Roussel-Philippe et al., European Journal of Lipid Science and Technology 102[2], 97-101. 2000): prior to adding the lauryl chloride to the wheat protein hydrolysate (10% by weight in water), a pH of 9 was established by adding NaOH. Then, at a temperature of 4° C., lauryl chloride was added stepwise up to a concentration of 0.625% by weight. After 4 hours, the pH was adjusted to 5 by adding HCl in order to hydrolyse possibly unreacted acid chloride to the fatty acid. The pH was then adjusted to 7.5 by adding NaOH. The foam volume and the foam stability of the alkylamine- and of the acid chloride-modified wheat protein hydrolysate were compared against one another as described in Example 3. The samples were adjusted to a protein content of 1% by weight for the experiment. In the case of the alkylamine-modified wheat protein hydrolysate, a greater foam formation was observed (15 ml instead of 10 ml in the case of the acid chloride-modified one).

1. An alkylated peptide mixture produced by a process comprising:
A) hydrolyzing at least one protein comprising at least one glutamine radical to produce a protein hydrolysate; and
B) contacting the protein hydrolysate with a transglutaminase and at least one primary or secondary amine of the general formula (I) 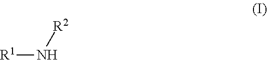
wherein each of R¹ and R² is independently selected from hydrogen atom and saturated or unsaturated, substituted or unsubstituted, branched or unbranched organic radicals having 5 to 40 carbon atoms, to produce said alkylated peptide mixture.

2. The alkylated peptide mixture according to claim 1, wherein said at least one protein is selected from the group consisting of isolated plant storage proteins, animal proteins, and microbial proteins.

3. The alkylated peptide mixture according to claim 1, wherein the hydrolysis in process step A) is catalysed by at least one acid or at least one enzyme.

4. The alkylated peptide mixture according to claim 1, wherein said protein hydrolysate in process step A) has an average molecular weight of 203 g/mol to 100,000 g/mol.

5. The alkylated peptide mixture according claim 1, wherein said protein hydrolysate in process step A) is selected from Meripro 810, Meripro 711, Naturalys W, Cropeptide W, Hydrotriticum 2000, Tritisol, Tritisol XM, Hydrosoy 2000, Gluadin W20, Gluadin WLM, AMCO HCA411, and HLA-198.

6. The alkylated peptide mixture according to claim 1, wherein said transglutaminase in process step B) is isolated from Bacillus subtilis or Streptoverticillium mombaraensis.

7. The alkylated peptide mixture according to claim 1, wherein said radicals R¹ and R² are independently selected from the group consisting of alkyl and alkenyl radicals.

8. The alkylated peptide mixture according to claim 1, wherein, in process step A), the hydrolysis is catalysed by at least one enzyme, and process step A) and process step B) are conducted simultaneously.

9. A process for the preparation of an alkylated peptide mixture, the process comprising:
A) hydrolyzing at least one protein comprising at least one glutamine radical to produce a protein hydrolysate; and
B) contacting the protein hydrolysate with a transglutaminase and at least one primary or secondary amine of the general formula (I) 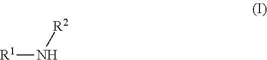
wherein each of R¹ and R² is independently selected from hydrogen atom and saturated or unsaturated, substituted or unsubstituted, branched or unbranched organic radicals having 5 to 40 carbon atoms, to produce said alkylated peptide mixture.

10. A cosmetic, dermatological, pharmaceutical crop protection, care, cleaning, or surfactant formulation comprising at least one alkylated peptide mixture according to claim 1.

11. An emulsifier, dispersion auxiliary, conditioner for skin and hair, foam former, or foam stabiliser comprising at least one alkylated peptide mixture according to claim 1.

12. The alkylated peptide mixture according to claim 1, further comprising purification of the protein hydrolysate in step (A).

13. The alkylated peptide mixture according to claim 1, further comprising purifying the alkylated peptide mixture.

14. The alkylated peptide mixture according to claim 7, wherein said radicals R¹ and R² are independently selected from the group consisting of linear unsubstituted alkyl and alkenyl radicals.

15. The alkylated peptide mixture according to claim 14, wherein said linear unsubstituted alkyl and alkenyl radicals are selected from the group consisting of octyl, decyl, dodecyl, tetradecyl, hexadecyl, octadecyl, octadecenyl, octadecadienyl, eicosyl, and docosyl radicals.

16. The method according to claim 9, further comprising purification of the protein hydrolysate in step (A).

17. The method according to claim 9, further comprising purifying the alkylated peptide mixture.

18. The method according to claim 9, wherein said radicals R¹ and R² are independently selected from the group consisting of linear unsubstituted alkyl and alkenyl radicals.

19. The method according to claim 18, wherein said linear unsubstituted alkyl and alkenyl radicals are selected from the group consisting of octyl, decyl, dodecyl, tetradecyl, hexadecyl, octadecyl, octadecenyl, octadecadienyl, eicosyl, and docosyl radicals.
